(19) United States
(12) Patent Application Publication
(10) Pub. No.: US 2024/0074681 A1
(43) Pub. Date: Mar. 7, 2024

(54) ANTENNA ARRAYS FOR ANALYTE SENSORS

(71) Applicant: Know Labs, Inc., Seattle, WA (US)

(72) Inventor: Phillip BOSUA, Seattle, WA (US)

(21) Appl. No.: 17/930,137

(22) Filed: Sep. 7, 2022

(51) Int. Cl. A61B 5/145

(52) U.S. Cl. CPC A61B 5/14546; A61B 5/14503; A61B 5/14532; H01Q 1/38

(57) ABSTRACT

A non-invasive analyte sensor includes a first antenna set and a second antenna set. The first antenna set includes a first antenna and a second antenna, and the second antenna set includes a third antenna (and optionally a fourth antenna). The first antenna set and the second antenna set are spaced apart from one another by a first distance with no antennas between the first antenna set and the second antenna set, the first antenna and the second antenna are spaced apart from one another by a second distance, and the first distance is greater than the second distance. In addition, the first antenna has an upper surface that faces in a first direction and the third antenna has an upper surface that faces in a second direction, and the first direction diverges away from the second direction.

### FIELD

This disclosure relates generally to apparatus, systems and methods of detecting an analyte via spectroscopic techniques using an analyte sensor that operates in the radio or microwave frequency range of the electromagnetic spectrum.

### BACKGROUND

There is interest in being able to detect and/or measure an analyte within a target. Sensors that use radio or microwave frequency bands of the electromagnetic spectrum for collection of analyte data are disclosed in WO 2019/217461, U.S. Pat. Nos. 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, each of which is incorporated herein by reference in its entirety.

### SUMMARY

This disclosure relates generally to apparatus, systems and methods of detecting an analyte via spectroscopic techniques using frequencies in the radio or microwave frequency range of the electromagnetic spectrum. A non-invasive analyte sensor described herein includes an antenna array (also referred to as a detector array) having a plurality of antennas (also referred to as detector elements or antenna elements) at least one of which can transmit an electromagnetic signal in the radio or microwave frequency range and at least one of which can receive an electromagnetic signal in the radio or microwave frequency range resulting from transmission of the electromagnetic signal.

In an embodiment, the antennas of the antenna array are arranged relative to one another to enhance detection performance while minimizing the size of the antenna array and thus minimizing the size of the analyte sensor. The antenna array can include 3 or 4 individual antennas that comprise longitudinal strips of a metal material or other material that can transmit and/or receive signals in the radio or microwave frequency range of the electromagnetic spectrum.

In one embodiment of the antenna array described herein, the antennas are disposed on a substrate (or multiple substrates) where the substrate(s) is shaped so that each antenna is more appropriately positioned relative to a target to improve the detection performance. For example, in the case of a human target, for example performing analyte detection on the palm of a person's hand using the analyte sensor, the substrate(s) supporting the antennas may be shaped so that during use of the analyte sensor, each one of the antennas may be located approximately the same distance from the palm. For example, the substrate(s) may be angled or curved/arcuate.

In one embodiment described herein, a non-invasive analyte sensor includes a first antenna set and a second antenna set. The first antenna set includes a first antenna and a second antenna, and the second antenna set includes a third antenna (and optionally a fourth antenna). The first antenna, the second antenna, and the third antenna each comprises an elongated strip of conductive material with a longitudinal axis. The longitudinal axes of the first antenna, the second antenna, and the third antenna may be parallel, substantially parallel, or generally parallel to each other. In addition, the first antenna set and the second antenna set are spaced apart from one another by a first distance with no antennas between the first antenna set and the second antenna set, the first antenna and the second antenna are spaced apart from one another by a second distance, and the first distance is greater than the second distance.

In another embodiment described herein, a non-invasive analyte sensor includes a first antenna set and a second antenna set. The first antenna set includes a first antenna and a second antenna, and the second antenna set includes a third antenna (and optionally a fourth antenna). The first antenna, the second antenna, and the third antenna each comprises an elongated strip of conductive material with a longitudinal axis. The longitudinal axes of the first antenna, the second antenna, and the third antenna may be parallel, substantially parallel or generally parallel to each other. The first antenna set and the second antenna set are spaced apart from one another with no antennas between the first antenna set and the second antenna set. In addition, the first antenna has an upper surface that faces in a first direction, and the third antenna has an upper surface that faces in a second direction, and the first direction diverges away from the second direction. The second antenna also has an upper surface that also faces in a direction that is generally similar to the first direction.

### DRAWINGS

FIG. 1 is a schematic depiction of a non-invasive analyte sensor system with a non-invasive analyte sensor relative to a target according to an embodiment.

FIG. 2 illustrates an example of an antenna array described herein that can be used in the non-invasive analyte sensor.

FIG. 3 illustrates another example of an antenna array described herein that can be used in the non-invasive analyte sensor.

FIG. 4 is an end view of the antenna array depicted in FIG. 2 in the direction of the line 4-4 in FIG. 2.

FIG. 5 is an end view of another embodiment of an antenna array.

FIG. 6 is an end view of another embodiment of an antenna array.

FIG. 7 is an end view of another embodiment of an antenna array.

FIG. 8 is an end view of another embodiment of an antenna array.

### DETAILED DESCRIPTION

The following is a detailed description of apparatus, systems and methods of detecting an analyte via spectroscopic techniques using frequencies in the radio or microwave frequency bands of the electromagnetic spectrum. An analyte sensor described herein includes an antenna array (also referred to as a detector array) having a plurality of antennas (also referred to as detector elements or antenna elements) at least one of which can transmit an electromagnetic signal in the radio or microwave frequency range into a target and at least one of which can receive an electromagnetic signal in the radio or microwave frequency range resulting from transmission of the electromagnetic signal into the target. The analyte sensor described herein operates by transmitting an electromagnetic signal in the radio or microwave frequency range of the electromagnetic spectrum toward and into a target using at least one transmit antenna. A returning signal that results from the transmission of the transmitted signal is detected by at least one receive antenna. The signal(s) detected by the receive antenna(s) can be analyzed to detect the analyte based on the intensity of the received signal(s) and reductions in intensity at one or more frequencies where the analyte absorbs the transmitted signal.

The analyte sensor described herein can be described as being non-invasive meaning that the sensor remains outside the target, such as the human body, and the detection of the analyte occurs without requiring removal of fluid or other removal from the target, such as the human body, although the non-invasive analyte sensor may be used to detect one or more analytes in fluid or other material that has been removed from the target. In the case of sensing an analyte in the human body, this non-invasive sensing may also be referred to as in vivo sensing.

The analyte sensor is configured to transmit generated transmit signals that are in a radio or microwave frequency range of the electromagnetic spectrum from the antenna(s) into a target containing an analyte, and to also detect responses that result from transmission of the transmit signals into the target. Examples of detecting analytes using non-invasive spectroscopy sensors operating in the radio or microwave frequency range of the electromagnetic spectrum are described in WO 2019/217461, U.S. Pat. Nos. 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, the entire contents of each are incorporated herein by reference.

In one embodiment, the sensor systems described herein can be used to detect the presence of at least one analyte in a target. In another embodiment, the sensor systems described herein can detect an amount or a concentration of the at least one analyte in the target. The target can be any target containing at least one analyte of interest that one may wish to detect. The target can be human or non-human, animal or non-animal, biological or non-biological. For example, the target can include, but is not limited to, human tissue, animal tissue, plant tissue, an inanimate object, soil, a fluid, genetic material, or a microbe. Non-limiting examples of targets include, but are not limited to, one or more of blood, interstitial fluid, cerebral spinal fluid, lymph fluid or urine, human tissue, animal tissue, plant tissue, an inanimate object, soil, genetic material, or a microbe.

The analyte(s) can be any analyte that one may wish to detect. The analyte can be human or non-human, animal or non-animal, biological or non-biological. For example, the analyte(s) can include, but is not limited to, one or more of glucose, blood alcohol, oxygen, white blood cells, or luteinizing hormone. The analyte(s) can include, but is not limited to, a chemical, a combination of chemicals, a virus, a bacteria, or the like. The analyte can be a chemical included in another medium, with non-limiting examples of such media including a fluid containing the at least one analyte, for example blood, interstitial fluid, cerebral spinal fluid, lymph fluid or urine, human tissue, animal tissue, plant tissue, an inanimate object, soil, genetic material, or a microbe. The analyte(s) may also be a non-human, non-biological particle such as a mineral or a contaminant.

The analyte(s) can include, for example, naturally occurring substances, artificial substances, metabolites, and/or reaction products. As non-limiting examples, the at least one analyte can include, but is not limited to, insulin, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyro sine, tryptophan); andrenostenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free (3-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, polio virus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; zinc protoporphyrin; prostaglandins such as PGF2α and PGE2; hormones such as estrogen, progesterone, and/or follicle stimulating hormone (FSH).

The analyte(s) can also include one or more chemicals introduced into the target. The analyte(s) can include a marker such as a contrast agent, a radioisotope, or other chemical agent. The analyte(s) can include a fluorocarbon-based synthetic blood. The analyte(s) can include a drug or pharmaceutical composition, with non-limiting examples including ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The analyte(s) can include other drugs or pharmaceutical compositions. The analyte(s) can include neurochemicals or other chemicals generated within the body, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

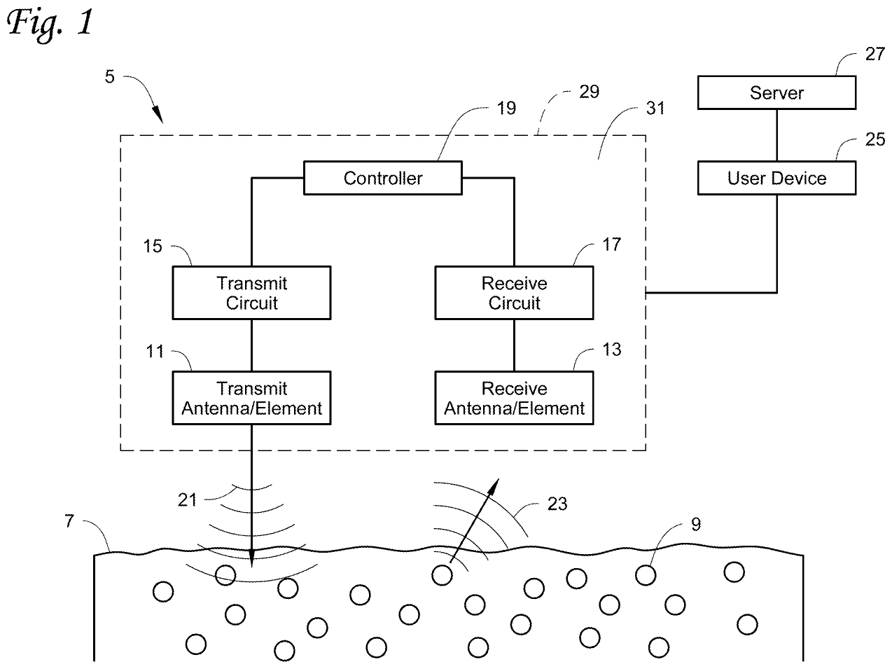

Referring now to FIG. 1, an embodiment of a non-invasive analyte sensor system with a non-invasive analyte sensor 5 is illustrated. The sensor 5 is depicted relative to a target 7 that contains an analyte of interest 9, for example an analyte in interstitial fluid and/or blood in a human body. In this example, the sensor 5 is depicted as including an antenna array that includes a transmit antenna/element 11 (hereinafter “transmit antenna 11”) and a receive antenna/element 13 (hereinafter “receive antenna 13”). The sensor 5 further includes a transmit circuit 15, a receive circuit 17, and a controller 19. As discussed further below, the sensor 5 can also include a power supply, such as a battery (not shown in FIG. 1). In some embodiments, power can be provided from mains power, for example by plugging the sensor 5 into a wall socket via a cord connected to the sensor 5.

The transmit antenna 11 is positioned, arranged and configured to transmit a signal 21 that is the radio frequency (RF) or microwave range of the electromagnetic spectrum into the target 7. The transmit antenna 11 can be an electrode or any other suitable transmitter of electromagnetic signals in the radio frequency (RF) or microwave range. The transmit antenna 11 can have any arrangement and orientation relative to the target 7 that is sufficient to allow the analyte sensing to take place. In one non-limiting embodiment, the transmit antenna 11 can be arranged to face in a direction that is substantially toward the target 7.

The signal 21 transmitted by the transmit antenna 11 is generated by the transmit circuit 15 which is electrically connectable to the transmit antenna 11. The transmit circuit 15 can have any configuration that is suitable to generate a transmit signal to be transmitted by the transmit antenna 11. Transmit circuits for generating transmit signals in the RF or microwave frequency range are well known in the art. In one embodiment, the transmit circuit 15 can include, for example, a connection to a power source, a frequency generator, and optionally filters, amplifiers or any other suitable elements for a circuit generating an RF or microwave frequency electromagnetic signal. In an embodiment, the signal generated by the transmit circuit 15 can have a frequency that is in the range from about 10 kHz to about 100 GHz. In another embodiment, the frequency can be in a range from about 300 MHz to about 6000 MHz. In an embodiment, the transmit circuit 15 can be configured to sweep through a range of frequencies that are within the range of about 10 kHz to about 100 GHz, or in another embodiment a range of about 300 MHz to about 6000 MHz.

The receive antenna 13 is positioned, arranged, and configured to detect one or more electromagnetic response signals 23 that result from the transmission of the transmit signal 21 by the transmit antenna 11 into the target 7 and impinging on the analyte 9. The receive antenna 13 can be an electrode or any other suitable receiver of electromagnetic signals in the radio frequency (RF) or microwave range. In an embodiment, the receive antenna 13 is configured to detect an electromagnetic signal having a frequency that is in the range from about 10 kHz to about 100 GHz, or in another embodiment a range from about 300 MHz to about 6000 MHz. The receive antenna 13 can have any arrangement and orientation relative to the target 7 that is sufficient to allow detection of the response signal(s) 23 to allow the analyte sensing to take place. In one non-limiting embodiment, the receive antenna 13 can be arranged to face in a direction that is substantially toward the target 7.

The receive circuit 17 is electrically connectable to the receive antenna 13 and conveys the received response from the receive antenna 13 to the controller 19. The receive circuit 17 can have any configuration that is suitable for interfacing with the receive antenna 13 to convert the electromagnetic energy detected by the receive antenna 13 into one or more signals reflective of the response signal(s) 23. The construction of receive circuits are well known in the art. The receive circuit 17 can be configured to condition the signal(s) prior to providing the signal(s) to the controller 19, for example through amplifying the signal(s), filtering the signal(s), or the like. Accordingly, the receive circuit 17 may include filters, amplifiers, or any other suitable components for conditioning the signal(s) provided to the controller 19.

The controller 19 controls the operation of the sensor 5. The controller 19, for example, can direct the transmit circuit 15 to generate a transmit signal to be transmitted by the transmit antenna 11. The controller 19 further receives signals from the receive circuit 17. The controller 19 can optionally process the signals from the receive circuit 17 to detect the analyte(s) 9 in the target 7. In one embodiment, the controller 19 may optionally be in communication with at least one external device 25 such as a user device and/or a remote server 27, for example through one or more wireless connections such as Bluetooth, wireless data connections such a 4G, 5G, LTE or the like, or Wi-Fi. If provided, the external device 25 and/or remote server 27 may process (or further process) the signals that the controller 19 receives from the receive circuit 17, for example to detect the analyte(s) 9. If provided, the external device 25 may be used to provide communication between the sensor 5 and the remote server 27, for example using a wired data connection or via a wireless data connection or Wi-Fi of the external device 25 to provide the connection to the remote server 27.

With continued reference to FIG. 1, the sensor 5 may include a sensor housing 29 (shown in dashed lines) that defines an interior space 31. Components of the sensor 5 may be attached to and/or disposed within the housing 29. For example, the transmit antenna 11 and the receive antenna 13 are attached to the housing 29. In some embodiments, the antennas 11, 13 may be entirely or partially within the interior space 31 of the housing 29. In some embodiments, the antennas 11, 13 may be attached to the housing 29 but at least partially or fully located outside the interior space 31. In some embodiments, the transmit circuit 15, the receive circuit 17 and the controller 19 are attached to the housing 29 and disposed entirely within the sensor housing 29.

The receive antenna 13 may be decoupled or detuned with respect to the transmit antenna 11 such that electromagnetic coupling between the transmit antenna 11 and the receive antenna 13 is reduced. The decoupling of the transmit antenna 11 and the receive antenna 13 increases the portion of the signal(s) detected by the receive antenna 13 that is the response signal(s) 23 from the target 7, and minimizes direct receipt of the transmitted signal 21 by the receive antenna 13. The decoupling of the transmit antenna 11 and the receive antenna 13 results in transmission from the transmit antenna 11 to the receive antenna 13 having a reduced forward gain (S 21) and an increased reflection at output (S 22) compared to antenna systems having coupled transmit and receive antennas.

In an embodiment, coupling between the transmit antenna 11 and the receive antenna 13 is 95% or less. In another embodiment, coupling between the transmit antenna 11 and the receive antenna 13 is 90% or less. In another embodiment, coupling between the transmit antenna 11 and the receive antenna 13 is 85% or less. In another embodiment, coupling between the transmit antenna 11 and the receive antenna 13 is 75% or less.

Any technique for reducing coupling between the transmit antenna 11 and the receive antenna 13 can be used. For example, the decoupling between the transmit antenna 11 and the receive antenna 13 can be achieved by one or more intentionally fabricated configurations and/or arrangements between the transmit antenna 11 and the receive antenna 13 that is sufficient to decouple the transmit antenna 11 and the receive antenna 13 from one another.

For example, in one embodiment described further below, the decoupling of the transmit antenna 11 and the receive antenna 13 can be achieved by intentionally configuring the transmit antenna 11 and the receive antenna 13 to have different geometries from one another. Intentionally different geometries refers to different geometric configurations of the transmit and receive antennas 11, 13 that are intentional. Intentional differences in geometry are distinct from differences in geometry of transmit and receive antennas that may occur by accident or unintentionally, for example due to manufacturing errors or tolerances.

Another technique to achieve decoupling of the transmit antenna 11 and the receive antenna 13 is to provide appropriate spacing between each antenna 11, 13 that is sufficient to decouple the antennas 11, 13 and force a proportion of the electromagnetic lines of force of the transmitted signal 21 into the target 7 thereby minimizing or eliminating as much as possible direct receipt of electromagnetic energy by the receive antenna 13 directly from the transmit antenna 11 without traveling into the target 7. The appropriate spacing between each antenna 11, 13 can be determined based upon factors that include, but are not limited to, the output power of the signal from the transmit antenna 11, the size of the antennas 11, 13, the frequency or frequencies of the transmitted signal, and the presence of any shielding between the antennas. This technique helps to ensure that the response detected by the receive antenna 13 is measuring the analyte 9 and is not just the transmitted signal 21 flowing directly from the transmit antenna 11 to the receive antenna 13. In some embodiments, the appropriate spacing between the antennas 11, 13 can be used together with the intentional difference in geometries of the antennas 11, 13 to achieve decoupling.

In one embodiment, the transmit signal (or each of the transmit signals) can be transmitted over a transmit time that is less than, equal to, or greater than about 300 ms. In another embodiment, the transmit time can be less than, equal to, or greater than about 200 ms. In still another embodiment, the transmit time can be less than, equal to, or greater than about 30 ms. The transmit time could also have a magnitude that is measured in seconds, for example 1 second, 5 seconds, 10 seconds, or more. In an embodiment, the same transmit signal can be transmitted multiple times, and then the transmit time can be averaged. In another embodiment, the transmit signal (or each of the transmit signals) can be transmitted with a duty cycle that is less than or equal to about 50%.

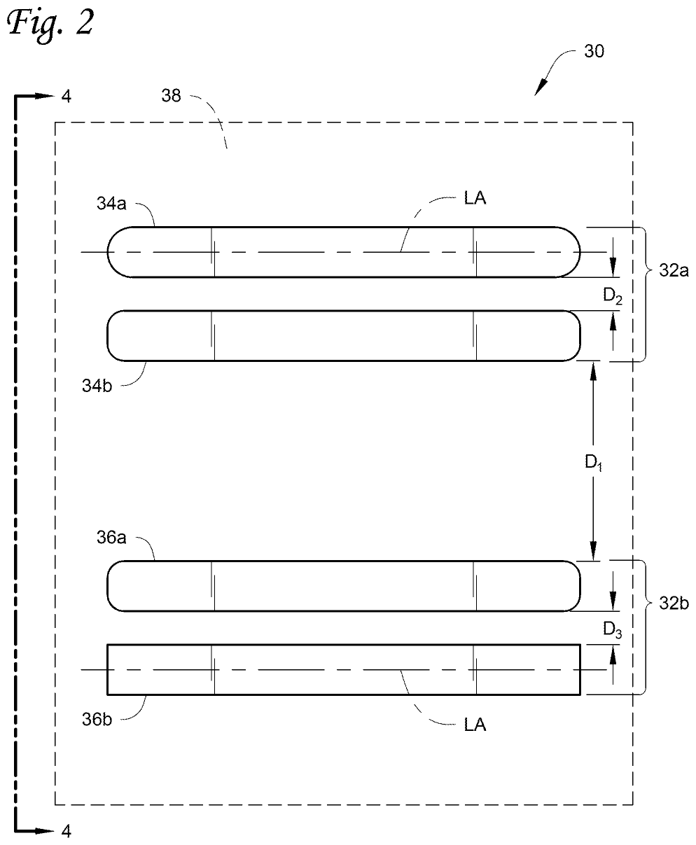

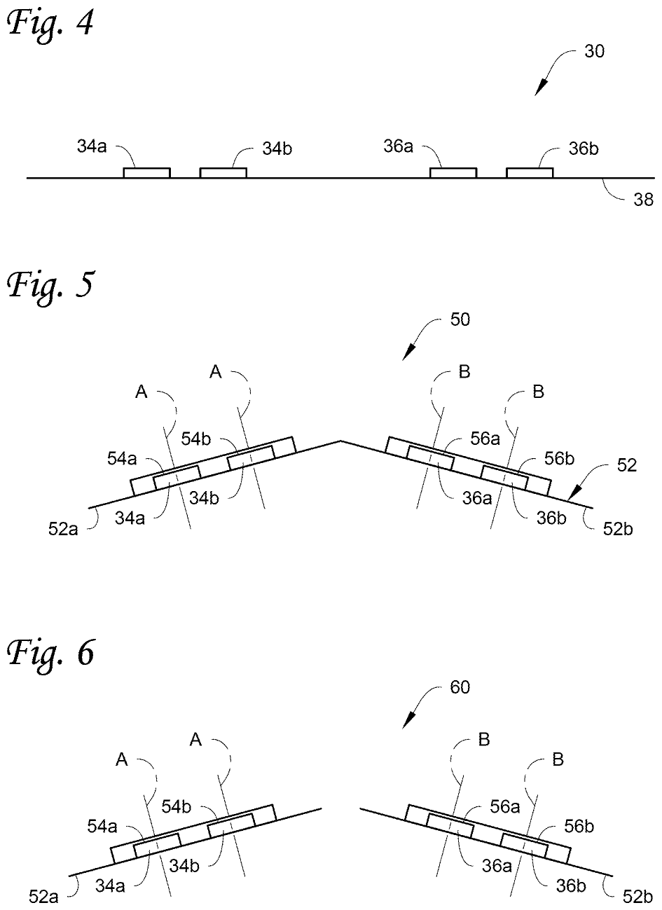

FIG. 2 illustrates an example of an antenna array 30 that can be used in the non-invasive analyte sensor 5 in place of the antennas 11, 13. The antenna array 30 is depicted as having a first antenna set 32a and a second antenna set 32b. The first antenna set 32a includes a first antenna 34a and a second antenna 34b, and the second antenna set 32b includes a third antenna 36a and a fourth antenna 36b. The first antenna 34a, the second antenna 34b, the third antenna 36a, and the fourth antenna 36b each comprises an elongated strip of conductive material with a longitudinal axis LA. The longitudinal axes LA of the antennas 34a, 34b, 36a, 36b may be parallel to one another, substantially parallel to one another, or generally parallel to one another with a slight angle between one or more of the longitudinal axes LA of one or more of the antennas 34a, 34b, 36a, 36b. As shown in FIGS. 2 and 4, the antennas 34a, 34b, 36a, 36b are depicted on a common substrate 38 which may be planar as shown in FIG. 4. However, the antennas 34a, 34b, 36a, 36b may be disposed on separate planar substrates, angled substrate(s), curved substrate(s), or other substrates. The antennas 34a, 34b, 36a, 36b are depicted in FIG. 4 as disposed on and projecting above the surface of the substrate 38. However, the antennas 34a, 34b, 36a, 36b can have other arrangements relative to the substrate such as being flush with the surface of the substrate 38.

Referring to FIG. 2, there are no antennas between the first antenna set 32a and the second antenna set 32b. The first antenna set 32a and the second antenna set 32b are spaced apart from one another by a first distance D₁. The first distance D₁ may be defined between the facing edges of the antennas 34b, 36a. In addition, the first antenna 34a and the second antenna 34b are spaced apart from one another by a second distance D₂ which may be defined between the facing edges of the antennas 34a, 34b. Similarly, the third antenna 36a and the fourth antenna 36b are spaced apart from one another by a third distance D₃ which may be defined between the facing edges of the antennas 36a, 36b. The first distance D₁ is greater than the second distance D₂ and the third distance D₃. The second distance D₂ may be equal to the third distance D₃, although in another embodiment the distances D₂ and D₃ may differ from one another.

As depicted in FIG. 2, some of the antennas 34a, 34b, 36a, 36b have different geometries from one another. For example, at least three of the antennas 34a, 34b, 36a, 36b have geometries that differ from one another. For example, at least one of the antennas 34a, 34b, 36a, 36b has a rectangular shape, at least one of the antennas 34a, 34b, 36a, 36b has a stadium shape, and at least one of the antennas 34a, 34b, 36a, 36b has a rounded rectangle shape. In the illustrated embodiment, the antenna 34a has a stadium shape, the antennas 34b, 36a have a rounded rectangle shape, and the antenna 36b has a rectangular shape. However, other combinations and arrangements of shapes for the antennas 34a, 34b, 36a, 36b can be used. A stadium shape is a two-dimensional geometric shape constructed of a rectangle with semicircles at opposite ends. A rounded rectangle shape is a two-dimensional geometric shape constructed of a rectangle with radiuses at each corner of the rectangle. The antennas in FIG. 2 are arranged on the substrate 38 in a manner such that two antennas with the same shape are not located next to one another.

With the shapes depicted in FIG. 2, each of the antennas 34a, 34b, 36a, 36b have a first longitudinal end and a second longitudinal end. The first longitudinal end of the antenna 34a has a geometry that differs from geometries of the first longitudinal ends of at least two or more of the antennas 34b, 36a, 36b, and the second longitudinal end of the antenna 34a has a geometry that differs from geometries of the second longitudinal ends of at least two or more of the antennas 34b, 36a, 36b. Similarly, the first longitudinal end of the antenna 34b has a geometry that differs from geometries of the first longitudinal ends of at least two or more of the antennas 34a, 36a, 36b, and the second longitudinal end of the antenna 34b has a geometry that differs from geometries of the second longitudinal ends of at least two or more of the antennas 34a, 36a, 36b. Similarly, the first longitudinal end of the antenna 36a has a geometry that differs from geometries of the first longitudinal ends of at least two or more of the antennas 34a, 34b, 36b, and the second longitudinal end of the antenna 36a has a geometry that differs from geometries of the second longitudinal ends of at least two or more of the antennas 34a, 34b, 36b. Similarly, the first longitudinal end of the antenna 36b has a geometry that differs from geometries of the first longitudinal ends of at least two or more of the antennas 34a, 34b, 36a, and the second longitudinal end of the antenna 36b has a geometry that differs from geometries of the second longitudinal ends of at least two or more of the antennas 34a, 34b, 36a.

In an embodiment, any one or more of the antennas 34a, 34b, 36a, 36b can be connected to the transmit circuit 15 whereby any one or more of the antennas 34a, 34b, 36a, 36b can function as a transmit antenna. In addition, any one or more of the antennas 34a, 34b, 36a, 36b can be connected to the receive circuit 17 whereby any one or more of the antennas 34a, 34b, 36a, 36b can function as a receive antenna to detect a response that results from transmitting the transmit signal into the target. Techniques for connecting to any antennas to function as a transmit antenna and connecting to any antennas to function as a receive antenna are disclosed in U.S. Pat. No. 11,058,331 the entire contents of which are incorporated herein by reference.

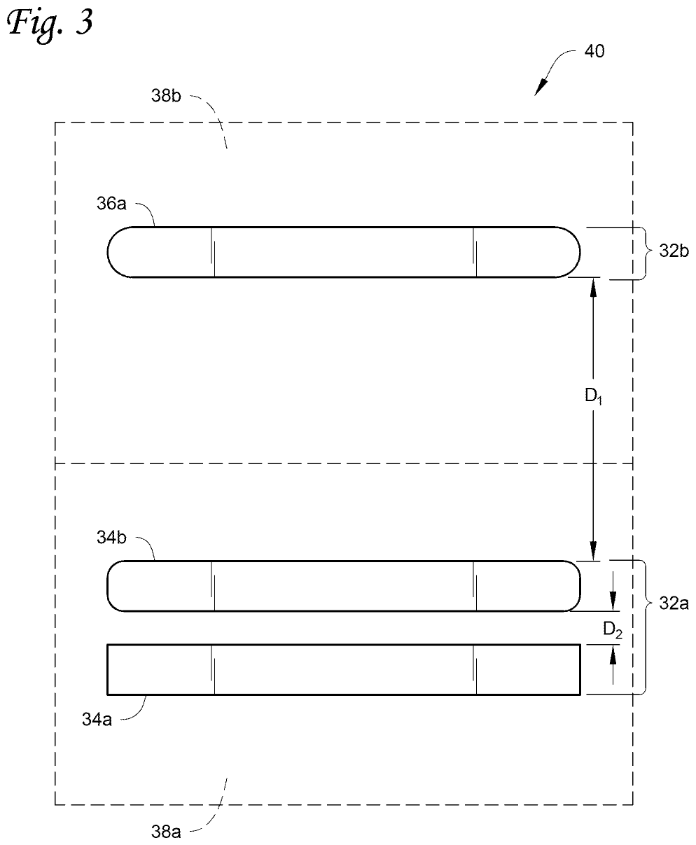

The embodiment depicted in FIG. 2 is illustrated as including four antennas, with each antenna set 32a, 32b including two antennas. However, a larger or smaller number of antennas can be used. For example, FIG. 3 illustrates another embodiment of an antenna array 40. In FIG. 3, elements that are the same as or similar to elements in FIG. 2 are referenced using the same reference numerals. FIG. 3 depicts the array 40 as including three antennas, with the antenna set 32a including two antennas and the antenna set 32b including a single antenna, with no antennas between the antenna sets 32a, 32b. In another embodiment, the antenna set 32a can include a single antenna while the antenna set 32b includes two antennas. In this example, the antenna 34a is depicted as having a rectangular shape, the antenna 34b is depicted as having a rounded rectangle shape, and the antenna 36a is depicted as having a stadium shape. However, other combinations and arrangements of shapes for the antennas 34a, 34b, 36a can be used.

In the example in FIG. 3, the first distance D 1 between the antenna sets 32a, 32b is greater than the second distance D₂ like in FIG. 2. In addition, instead of a common substrate 38 like in FIG. 2, the array 40 has separate substrates 38a, 38b, with the antenna set 32a mounted on a first substrate 38a and the antenna set 32b mounted on a second substrate 38b. The substrates 38a, 38b may each be planar, and they may be parallel to one another.

FIG. 5 is an end view of another embodiment of an antenna array 50. In FIG. 5, elements that are the same as or similar to elements in FIGS. 2 and 3 are referenced using the same reference numerals. In this embodiment, the antennas 34a, 34b of the first antenna set is disposed on a first angled portion 52a of a common substrate 52, and the antennas 36a, 36b of the second antenna set is disposed on a second angled portion 52b of the common substrate 52. The antennas 34a, 34b, 36a, 36b can have the same configuration as the antennas described above for FIGS. 2 and 3. Although the array 50 is depicted as having four antennas, the array 50 can have a different number of antennas, such as three antennas as illustrated in FIG. 3.

Upper surfaces 54a, 54b of the antennas 34a, 34b face in a first direction along an axis A, and upper surfaces 56a, 56b of the antennas 36a, 36b face in a second direction along an axis B. Due to the angle between the portions 52a, 52b of the substrate 52, the direction of the axis A diverges away from the direction of the axis B and vice versa.

FIG. 6 illustrates an embodiment of an antenna array 60 that is similar to the antenna array 50 and elements in FIG. 6 that are the same as or similar to elements in the array 50 are referenced using the same reference numerals. In the array 60, instead of the common substrate 52 depicted in FIG. 5, the angled portions 52a, 52b are separate from one another and form separate substrates for the antennas 34a, 34b, 36a, 36b.

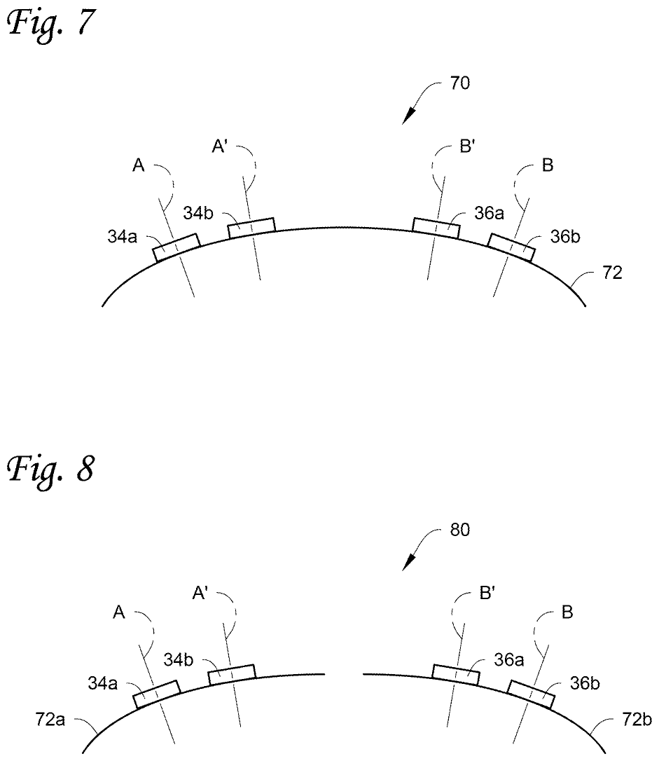

FIG. 7 is an end view of another embodiment of an antenna array 70. In FIG. 7, elements that are the same as or similar to elements in FIGS. 2, 3 and 5 are referenced using the same reference numerals. In this embodiment, the antennas 34a, 34b, 36a, 36b are disposed on a curved or arcuate common substrate 72. The antennas 34a, 34b, 36a, 36b can have the same configuration as the antennas described above for FIGS. 2 and 3. Although the array 70 is depicted as having four antennas, the array 70 can have a different number of antennas, such as three antennas as illustrated in FIG. 3.

Due to the curvature of the substrate 72, the upper surface 54a of the antenna 34a faces in the first direction along the axis A, and the upper surface 54b of the antenna 34b faces in a direction A′ that is slightly different than the direction of the axis A, but can be characterized as generally similar to the direction of the axis A. Similarly, the upper surface 56a of the antenna 36a faces in a direction along an axis B′ that is slightly different than the direction of the axis B of the upper surface 56b of the antenna 36b, but can be characterized as generally similar to the direction of the axis B. The axes A, A′ both diverge away from the axes B, B′ and vice versa.

FIG. 8 illustrates an embodiment of an antenna array 80 that is similar to the antenna array 70 and elements in FIG. 8 that are the same as or similar to elements in the array 70 are referenced using the same reference numerals. In the array 80, instead of the common substrate 72 depicted in FIG. 7, the antennas 34a, 34b are disposed on and supported by a first curved or arcuate substrate 72a, while the antennas 36a, 36b are disposed on and supported by a second curved or arcuate substrate 72b.

The terminology used in this specification is intended to describe particular embodiments and is not intended to be limiting. The terms “a,” “an,” and “the” include the plural forms as well, unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A non-invasive analyte sensor, comprising:
a first antenna set and a second antenna set;
the first antenna set includes a first antenna and a second antenna, and the second antenna set includes a third antenna;
the first antenna, the second antenna, and the third antenna each comprises an elongated strip of conductive material with a longitudinal axis;
the first antenna set and the second antenna set are spaced apart from one another by a first distance with no antennas between the first antenna set and the second antenna set, the first antenna and the second antenna are spaced apart from one another by a second distance, and the first distance is greater than the second distance.

2. The non-invasive analyte sensor of claim 1, wherein the longitudinal axes of the first antenna, the second antenna, and the third antenna are parallel to each other.

3. The non-invasive analyte sensor of claim 2, wherein the second antenna set further includes a fourth antenna that comprises an elongated strip of conductive material with a longitudinal axis, and the longitudinal axis of the fourth antenna is parallel to the longitudinal axes of the first antenna, the second antenna, and the third antenna; and
the third antenna and the fourth antenna are spaced apart from one another by a third distance, and the third distance equals the second distance.

4. The non-invasive analyte sensor of claim 3, wherein at least three of the first antenna, the second antenna, the third antenna, and the fourth antenna have geometries that differ from one another.

5. The non-invasive analyte sensor of claim 3, wherein each of the first antenna, the second antenna, the third antenna and the fourth antenna have a first longitudinal end and a second longitudinal end; the first longitudinal end of the first antenna has a geometry that differs from geometries of the first longitudinal ends of at least two or more of the second antenna, the third antenna and the fourth antenna; and the second longitudinal end of the first antenna has a geometry that differs from geometries of the second longitudinal ends of at least two or more of the second antenna, the third antenna and the fourth antenna.

6. The non-invasive analyte sensor of claim 3, wherein at least one of the first antenna, the second antenna, the third antenna and the fourth antenna has a rectangular shape; at least one of the first antenna, the second antenna, the third antenna and the fourth antenna has a stadium shape; and at least one of the first antenna, the second antenna, the third antenna and the fourth antenna has a rounded rectangle shape.

7. The non-invasive analyte sensor of claim 1, further comprising:
a transmit circuit that is configured to generate a transmit signal to be transmitted, the transmit signal is in a radio or microwave frequency range of the electromagnetic spectrum; and the transmit circuit is electrically connectable to any one or more of the first antenna, the second antenna and the third antenna whereby any one or more of the first antenna, the second antenna and the third antenna can function as a transmit antenna; and
a receive circuit that is electrically connectable to any one or more of the first antenna, the second antenna and the third antenna whereby any one or more of the first antenna, the second antenna and the third antenna can function as a receive antenna to detect a response that results from transmitting the transmit signal into a target.

8. The non-invasive analyte sensor of claim 1, wherein the first antenna set and the second antenna set are disposed on a common substrate.

9. A non-invasive analyte sensor, comprising:
a first antenna set and a second antenna set;
the first antenna set includes a first antenna and a second antenna, and the second antenna set includes a third antenna;
the first antenna, the second antenna, and the third antenna each comprises an elongated strip of conductive material with a longitudinal axis;
the first antenna set and the second antenna set are spaced apart from one another with no antennas between the first antenna set and the second antenna set; and
the first antenna has an upper surface that faces in a first direction, the third antenna has an upper surface that faces in a second direction, and the first direction diverges away from the second direction.

10. The non-invasive analyte sensor of claim 9, wherein the longitudinal axes of the first antenna, the second antenna, and the third antenna are parallel to each other.

11. The non-invasive analyte sensor of claim 10, wherein the second antenna set further includes a fourth antenna that comprises an elongated strip of conductive material with a longitudinal axis, and the longitudinal axis of the fourth antenna is parallel to the longitudinal axes of the first antenna, the second antenna, and the third antenna; and
the fourth antenna has an upper surface that faces in the second direction.

12. The non-invasive analyte sensor of claim 11, wherein at least three of the first antenna, the second antenna, the third antenna and the fourth antenna have geometries that differ from one another.

13. The non-invasive analyte sensor of claim 11, wherein each of the first antenna, the second antenna, the third antenna and the fourth antenna have a first longitudinal end and a second longitudinal end; the first longitudinal end of the first antenna has a geometry that differs from geometries of the first longitudinal ends of at least two or more the second antenna, the third antenna and the fourth antenna; and the second longitudinal end of the first antenna has a geometry that differs from geometries of the second longitudinal ends of at least two or more of the second antenna, the third antenna and the fourth antenna.

14. The non-invasive analyte sensor of claim 11, wherein at least one of the first antenna, the second antenna, the third antenna and the fourth antenna has a rectangular shape; at least one of the first antenna, the second antenna, the third antenna and the fourth antenna has a stadium shape; and at least one of the first antenna, the second antenna, the third antenna and the fourth antenna has a rounded rectangle shape.

15. The non-invasive analyte sensor of claim 9, further comprising:
a transmit circuit that is configured to generate a transmit signal to be transmitted, the transmit signal is in a radio or microwave frequency range of the electromagnetic spectrum; and the transmit circuit is electrically connectable to any one or more of the first antenna, the second antenna and the third antenna whereby any one or more of the first antenna, the second antenna and the third antenna can function as a transmit antenna; and
a receive circuit that is electrically connectable to any one or more of the first antenna, the second antenna and the third antenna whereby any one or more of the first antenna, the second antenna and the third antenna can function as a receive antenna to detect a response that results from transmitting the transmit signal into a target.

16. The non-invasive analyte sensor of claim 9, wherein the first antenna set and the second antenna set are disposed on a common substrate.

17. The non-invasive analyte sensor of claim 9, wherein the first antenna set is disposed on a first angled portion of a substrate, and the second antenna set is disposed on a second angled portion of a substrate.

18. The non-invasive analyte sensor of claim 16, wherein the common substrate is curved.
